(19) United States
(12) Patent Application Publication
(10) Pub. No.: US 2016/0054219 A1
(43) Pub. Date: Feb. 25, 2016

(54) Compact Size Explosives Detector with Ultra Fast Response and High Sensitivity and Method for Detecting Explosives

(71) Applicants: JIRI BLAHA, OBERÄGERI (CH); JAN DUPAC, PRAHA 4 (CZ); MICHAL ZÁSTERA, PRAHA 8 (CZ); ROMAN MÁZL, PRAHA 6 (CZ)

(72) Inventors: JIRI BLAHA, OBERÄGERI (CH); JAN DUPAC, PRAHA 4 (CZ); MICHAL ZÁSTERA, PRAHA 8 (CZ); ROMAN MÁZL, PRAHA 6 (CZ)

(73) Assignee: RS DYNAMICS GMBH, ZUG (CH)

(21) Appl. No.: 14/425,516

(22) PCT Filed: Sep. 3, 2012
(86) PCT No.: PCT/IB2012/001693

§ 371 Date: Nov. 12, 2015

(51) Int. Cl. G01N 21/3504; G01N 33/22

(52) U.S. Cl. CPC G01N 21/3504; G01N 33/227

(57) ABSTRACT

A system and methodology for semi-selective Infra-Red sampling and detection of explosive traces is described. The detection system combines the advanced Infra-Red sampling technique, capable to sample even non-volatile explosives in vapor mode with sensitive and interfering compounds extremely resistant analytical unit for reliable detection of all explosive compounds. All presented technology is designed to create ultra-miniature pocket-sized, ultra-fast detection system.

### FIELD OF THE INVENTION

This invention claims priority based on a compact size explosive detector with ultra fast response and high sensitivity.

This invention also relates to the method for the detecting explosives.

### BACKGROUND OF THE INVENTION

Current technologies used for detection of explosive traces are obviously based on direct air sampling of explosive vapors surrounding the found explosive contraband or particulate sampling technique. The direct air sampling is effective only for sufficiently volatile explosive components, while the explosive components, mostly used for creation of so called plastic explosives feature very low or zero volatility at ambient temperature.

There is a great demand world-wide for reliable contact-less detection technologies, being capable to sample and detect all kinds of explosive compounds, mainly widely used non-volatile or home-made explosive agents.

Most of the current detection technologies use an IMS (Ion Mobility Spectrometry) detection principle, or other technologies, focused for direct identification of the sample; unfortunately, those detection principles are very sensitive for cross-talks by various disturbing chemicals and saturation due to very limited dynamic range. Both above limitations create potentially high false alarm rate and causes moreover a number of serious problems with after-exposure cleaning of the system.

The current portable systems work mostly in the cycle “sampling-pre-concentration-analyses”. This operation cycle requires the operator to sample just from one spot, in the good believe this spot contains the found traces. There is no indication about the properly selected spot during the sampling interval, so it frequently happens to sample from the improper spot or location, while losing a valid operational time.

Most of the current systems, utilizing various detection technologies, namely IMS, are quite complicated, climatic conditions-sensitive instruments, not very suitable for heavy-duty field and/or military application. Also, they require advanced level of operator's qualification and demanding training of the operator.

Most of today's operators require to scan the controlled subjects (parcels, baggage, people) only in vapor mode, that is under standard sampling condition practically not successful namely in the case of plastic explosives (and all other non-volatile explosives), because those substances create no vapors at normal ambient temperature and that is why no vapor traces can be sampled/detected in standard vapor mode.

Systems and methods for reliable detection of various explosives are urgently needed and are now at the forefront of many research affords. The best wanted detection system should be extremely reliable, sensitive, the smallest sized, operating preferably only in vapor mode, resistant against cross-talking chemicals to avoid false alarms and very simple for operation not to require extensive training and special education of the operator. Under the standard environmental conditions, the detection of explosives is very complicated, complex task, especially if we consider detection of all branch of non-volatile types of explosives, disturbing factors of the variety of environmental condition (humidity, dust, temperature). Moreover, interferences from various chemicals, human sweat, various solvents, e.t.c. lead to false alarms, that are obviously difficult to distinguish from actual positive detection.

The wide variety of techniques, used to sample explosives include manual swiping of the scanned subject in so called particulate mode or sampling the air, surrounding the scanned subject at ambient temperature and consequent pre-concentration in a good believe to collect the most of the traces from the vicinity of the scanned subject. The manual swiping is not very popular or sometimes difficult to apply, air sampling requires huge amount of air to be sampled and pre-concentrated to collect enough volume of traces to satisfy the system detection limit.

The variety of techniques, used to detect/analyze explosives, based on ion mobility spectrometry, IR spectroscopy, micro-wave spectroscopy, Raman or fluorescence spectrometry bring good detection limits, however all those principles feature low dynamic range and are very sensitive to cross-talks, caused by interfering chemicals, overloads and suffer from difficult cleaning if exposed by huge sampled concentration. Portable detection system built on above principles are usually complicated, big, heavy and not operationally robust enough to work reliably in the demanding field conditions, characterized by changing the humidity, temperature, dust and rough handling by the operator. High level of operator's training and adequate operator's education is obviously necessary for successful detection using above technologies, that limits the range of people usable to make the detection job.

### SUMMARY OF THE INVENTION

The current invention relates to a system and methodology for ultra-fast, highly sensitive, continuous vapor-mode detection of explosives.

The present disclosure further relates to the technical design and sub-miniature construction of the system, using Infra-Red (further IR) Continuous Sampling technique, together with newly invented thermal decomposition detection unit, integrated-in one body with Infra-Red sampler.

In the new embodiment, the newly designed detection system, according to this invention, is built in very compact, pocket sized and light-weight body, especially shaped for the whole-day wearing on the policeman belt.

In another embodiment, the new system works in direct Infra-Red continuous Vapor Mode with instant reading, not requiring sampling, pre-concentration and analyzing period.

In yet another embodiment, the uses new flat IR source transmitting the IR radiation directly to the scanned surface for ideal release of all volatile and also non-volatile explosive traces.

In yet another embodiment, the IR sampler is integrated into one body with thermal decomposition unit for fast detection, minimizing the loses of the scanned traces volume and minimizing the battery energy consumption.

In yet another embodiment, the thermal decomposition unit, directly integrated into one body with IR radiation source, is composed by a silica-glass tube with on-surface integrated non-linear heating element to achieve minimal temperature gradient along the length of the thermal decomposition tube and precision regulation of the operating temperature.

In yet another embodiment, the thermal decomposition unit is housed into special thermal insulating cylinder for optimal shape of the temperature gradient and minimizing the temperature loses.

In yet another embodiment, the new detection cartridge with sunk molecular membrane is used for reliable and long-term stable detection of photons as a response to the decomposed explosive molecules coming into reaction with detection liquid.

In yet another embodiment, the shape of the detection cartridge is especially designed to achieve the widest possible range of 3-D working angels of the cartridge, to achieve the detection membrane to be sank under the detection liquid level within the widest operational angles.

In yet another embodiment, the gas output of the thermal decomposition unit is connected with the detection cartridge using especially designed needle line-up, enabling fast and reliable connection and/or change of the detector cartridge.

In yet another embodiment, the new detection cartridge consists of the main cartridge body, housing the special detection liquid, still sank detection polymer membrane, sensing cap, creating detection annular chamber for convolute circulation of the detected gas along the dry membrane surface together with proper connection to the connecting needles, and the glossy optical window for detecting photons, created at the wet side of the membrane to pass through the layer of the detection liquid and through this window to the optical single photon detection unit.

In yet another embodiment, the construction shape of the detection cartridge is designed for the tightest possible displacement and optical connection to the single-photon optical detection unit.

In yet another embodiment, the construction shape of the detection cartridge is designed for the one-movement easy cartridge deployment to the connection needles line-up and easy replacement of the used cartridge.

In yet another embodiment, the detection cartridge is equipped with special pulling handle for easy manipulation and removing that from it's slot.

In yet another embodiment, the detection cartridge is equipped with soft plastic optical shield to avoid penetration even of the weakest interfering light into the cartridge.

In yet another embodiment, the detection cartridge is inserted into the rectangular slot, created inside the one-body instrument frame, housing also newly developed single photon optical detection unit.

In yet another embodiment, the one-body instrument frame contains two slots—one for detection cartridge and the other one for the single-photon optical detection unit to secure minimal distance between photo sensing element of the single-photon optical detection unit and the detection cartridge optical window.

In yet another embodiment, the one-body instrument frame creates the holding slot with silent-block vibration insulation lodgement for the vibration pump installation

In yet another embodiment, the one-body instrument frame creates the mounting points for the 3-D montage of the 3-D Flex PC board, containing all the system electronics, communication connectors and battery connectors, all at one Flex PC board.

In yet another embodiment, the one-body instrument frame contains the mounting points for connecting the front analytical part containing Infra-Red continuous sampler and thermal decomposition detection unit.

In yet another embodiment, the one-body instrument frame contains one system mounting point for fixing the whole instrument formation into the rectangular-tube-shaped explosive detector case, using one screw only.

In another embodiment, the explosive detector case is made of high-density lightweight carbon-fiber plastic to achieve extreme durability and robustness.

In still another embodiment, the newly designed single-photon detection unit, according to this invention, is used to achieve the highest level of system sensitivity.

In still another embodiment, the new system is equipped with built-in Wi-Fi module, placed on common 3-D Flex PC board, for wire-less remote control and data transfer.

In still yet another embodiment, the powerful multi-processor built-in computer and operational software was designed to control all the operations and to achieve easy user friendly operation.

In still yet another embodiment, the only one button is used to operate all the system functions due to intelligent, user-friendly operational software.

In still yet another embodiment, the full graphic, color screen is used to provide transparent, user friendly communication and detection results to the operator

In still yet another embodiment, the newly developed software is used for easy wire-less remote control for robotic application

In still yet another embodiment, the newly developed software is used for remote Internet factory checking and first-level factory repair of the each system.

In still yet another embodiment, the special belt holster is designed for convenient and dexterous wearing/using the unit in the field.

In yet another embodiment, four special LiPol battery cells are used as the main system rechargeable battery, housed in robust carbon-fibers battery case following the shape of the whole unit.

The present disclosure further relates to the method for detecting explosives comprising: Infra-Red release of the solid explosive samples from the surface of the scanned subject using the dedicated wavelength of the Infra-Red radiation, thermal decomposition of the obtained gas sample, converting the decomposed molecule's concentration to the optical signal, analyzing the shape of time peak of the optical signal to distinguish interfering components from the signal of interest and to remove wide spectrum noise and signal fluctuations of various origin and analyzing the data obtained from above signal processing, displaying the data in numerical and/or graphic form on the color graphic screen and storing the data to the system flash disk.

The main advantage of this invention is the system and methodology for detection of explosives, more particularly using Infra-Red semi-selective sampling technique built into ultra-miniature pocket-sized, ultra-fast detection system. The analytical and detection device according to the invention is equipped with various ways to communicate with surroundings. For manufacturer set up, calibration and software update, the standard RS232 port (located at module 44) is provided, using the advantages of simplicity and reliability of the communication protocol. The customer can use either standard USB 2.0 port 44, with standard micro-USB connector and cable to connect the unit to PC, or built-in WiFi module 45 for convenient remote data transfer and/or full remote control operation and Internet remote communication.

Special remote control software for PC and also for mobile ANDROID platform, using the wire-less Wifi connection, have been developed for convenient application of the detection and analytical system according to the invention, for outdoor/indoor field robotic application according to the invention.

The test results prove, that the cost effective, sensitive enough and simple, very portable and fast detection and analytical system has been created for quantitative detection of explosive traces in the presence of other organic material by combining the intelligent Infra-Red sampling with fast and sensitive thermal decomposition and optical detection/analytical unit. The developed system according to the current invention, is technically simple and provides clear data of presence and concentration of the explosive materials not disturbed by presence of the strong concentration of cross-talking organic chemical components. Even if detailed chemical identification of the explosive root components is not provided, this simple, fast and pocked sized system finds number of customers who calls for easy to operate, very sensitive, light-weight and operationally robust detection/analytical system.

### BRIEF DESCRIPTION OF THE DRAWINGS

These and other features and advantages of the present invention will be better understood by reference to the following detailed description when considered in conjunction with the accompanying drawing wherein:

FIG. 1 provides a block diagram of an exemplary explosive detector and particularly the detection system with Infra-Red sampler, thermal decomposition unit, analytical unit, main processor and interface units with consequent control and data acquisition parts and power control and supply unit.

FIG. 2 provides the drawing of the composition of the Infra-Red sampler and thermal decomposition unit, together with control electronics creating thus one building block of the exemplary explosive detector according to the invention, where in the upper part of the FIG. 2 is the axonometrical view of the Infra-Red sampler with termal decomposition unit, together with control electronics, all in one formation, in the lower part of FIG. 2, there is the front view of the Infra-red sampler on the left side and cross-section A-A of the whole formation on the lower right side.

FIG. 3 provides drawing of the formation of all the other operational units of the exemplary explosive detector according to the invention, where on the upper part of the FIG. 3 there is the drawing of the whole for formation and in the lower part of the FIG. 3, there is a disassembled view of the whole formation, showing all the individual parts of the formation and their positioning.

FIG. 4 provides the drawing of the detector cartridge together with the single photon optical detection unit, in disassembled view.

FIG. 5 provides drawing of the flat Infra-Red radiation source helix-shaped element which areal shape securing the areal homogeneuous radiation of the Infra-Red energy. In the upper part of FIG. 5 there is the front view of the Infra-Red radiation source and at the lower part of FIG. 5, tghre is the axonometric view of the helix-shaped radiation element.

FIG. 6 provides the drawing of the cylindrical Infra-Red sampler mirror, providing the increase of the efficiency of the Infra-Red radiation, where in the upper part of FIG. 6. there is the view of the whole constellation of the Infra-red sampler and, in the lower part of FIG. 6 there is a view of assembled individual parts with the cone-shaped hood removed.

FIG. 7 provides the drawing of IR sampler calibration unit with five heat flow detectors.

FIG. 8 provides drawing of the instrument frame with multiple processor board, where on the upper part of FIG. 8, there is an axonometric view of the whole formation of instrument frame with multiple processor board, the middle part of the FIG. 8 shows the disassembled view of the mentioned formation and the lower part of the FIG. 8 shows the right side view of the assembled formation.

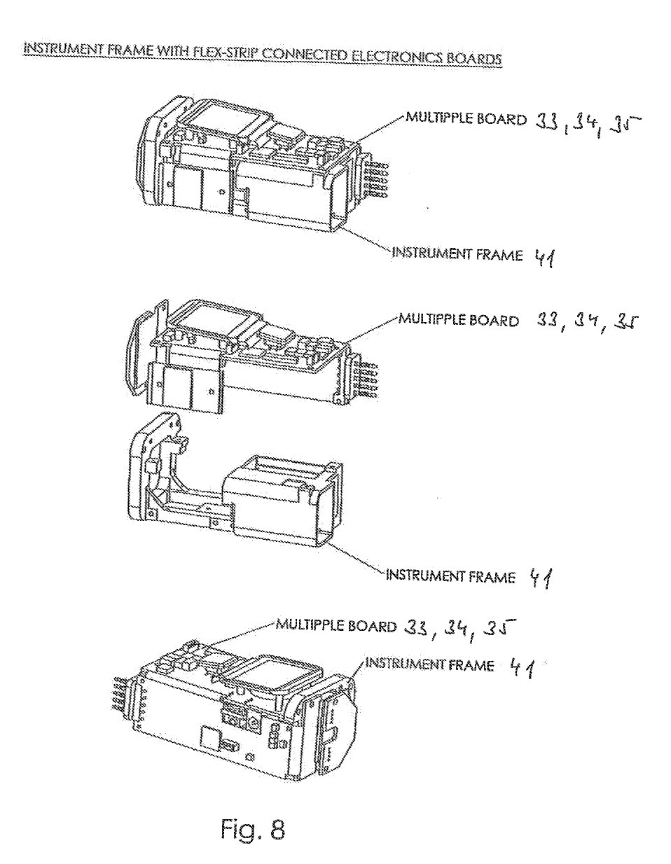

FIG. 9 shows the explosive detector carrying and protecting holster, where in the upper part of FIG. 9, there is a view of emptu holster ad in the lower part of FIG. 9, there is a view of the holster, carrying the explosive detector.

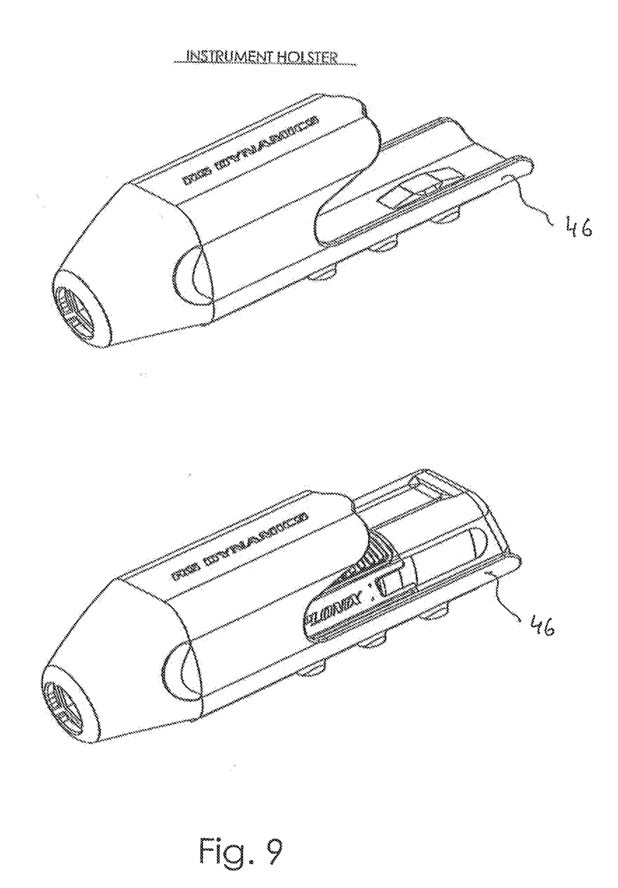

### DETAILED DESCRIPTION OF THE INVENTION EMBODIMENTS

Unless defined otherwise, all the technical and scientific terms used herein have the same meaning as commonly understood to one of ordinary skills in the art to which this invention belongs. Although any methods, devices and materials similar or equivalent to those described herein can be used in the practice or testing of the invention, the referred methods, devices and materials are described bellow.

Although conventional techniques have taken a multidimensional approach to explosive detection and analyses, obviously using gas chromatography followed by IMS detection unit or using the IMS system as the only detection part, these prior art techniques have been focused on addressing the unique problems in fast, continuous sampling and detection of namely non-volatile or very low vapor pressure explosives, using continuous vapor mode detection technique with Infra-Red sampling of explosive traces.

The current invention addresses the technical solution and operating methodology of the Infra-Red continuous mode sampler 1 together with integrated-in thermal decomposition unit 2 and consequent single photon optical detection unit 3 and intelligent data acquisition and control unit 4, communication unit 17 to create the ultra-fast, highly sensitive and false alarm extremely resistant explosive detection system, capable of instant sampling and detection of even non-volatile explosives in real-time instant vapor mode. The whole detection system, including all the sampling, detection and analytical components is designed with the aim to achieve the sub-miniature construction and low-cost high volume serial production to create affordable, simple to operate explosive detector for everyday use of the operator with no need of special qualification and/or training.

Sampling System

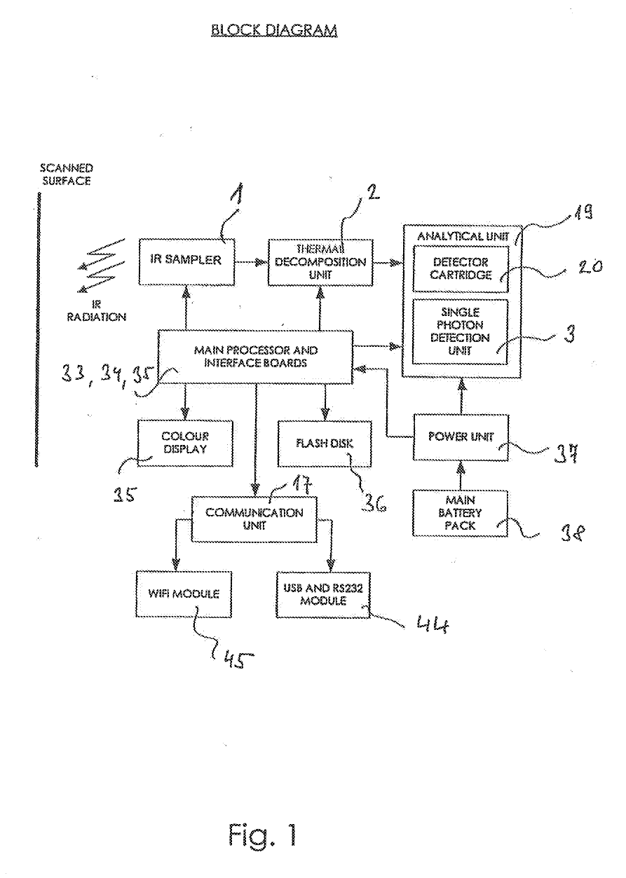

Turning to the sampling system, the new system according to the invention uses Infra-Red radiation energy of the selected wavelength to semi-selectively address explosive materials molecules, for their preferentially release from solid to vapor phase (FIG. 1).

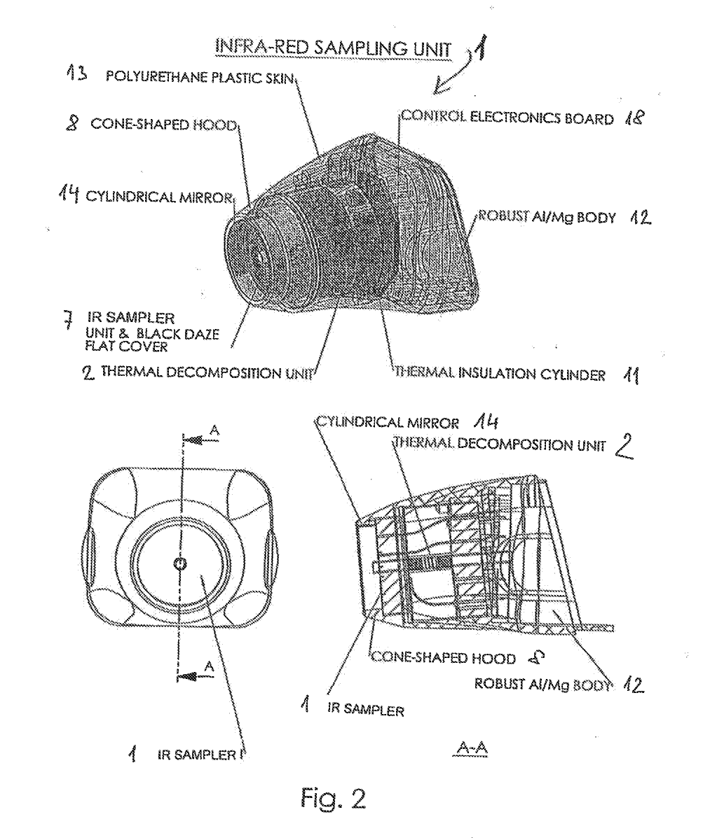

In exemplary embodiment, the sampling system consists of especially designed flat Infra-Red radiation source 5 (FIG. 2, 5), emitting the generated Infra-Red energy homogeneously from it's flat surface to the scanned surface.

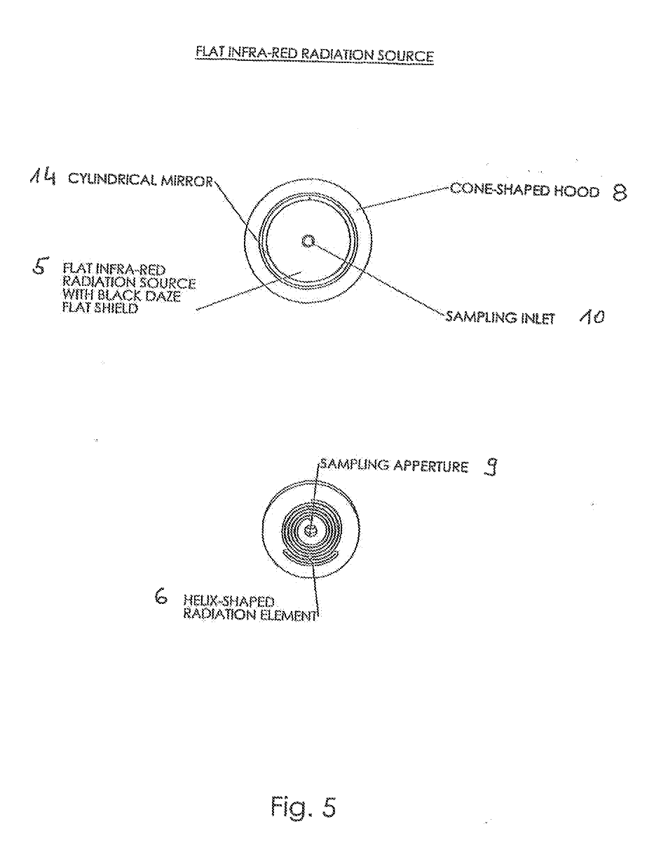

In another embodiment, the flat Infra-Red radiation source consists of the helix-shaped radiation element 6 (FIG. 5), with modified helix-shape, according to the invention, described on the FIG. 5, to achieve the maximal areal homogeneity of the radiated Infra-Red energy.

In the yet another embodiment, the flat Infra-Red radiation source 5 is covered with two-layers thin-film black daze shield (cover) 7 (FIG. 2) to allow mostly Infra-Red radiation to pass through the shield and, also, to protect the radiation source 5 against mechanical damage.

In yet another embodiment, the black daze flat Infra-Red source shield 7 is equipped with cone-shaped hood 8 (FIG. 5) for shielding the sampling process against the ambient blowing wind and the central circular aperture 9 (FIG. 5) for the sampling silica-glass inlet 10 to pass through.

In still yet another embodiment, the set of flat Infra-Red source 5, together with it's shield 7 according to the point [0066], is mechanically fixed at the temperature insulation cylinder with the central cylindrical room for the emplacement of the thermal decomposition unit 2. (FIG. 2).

In yet another embodiment, the set of Infra-Red radiation source 5, according to the points [0063-0066], is housed into the robust, molded aluminum/magnesium body 12 (FIG. 2) for achieving operational robustness and good temperature dissipation.

In yet another embodiment, the molded aluminum/magnesium body 12 is covered with polyurethane plastic skin 13 (FIG. 2) to avoid burning up the operator's fingers and to dump possible mechanical impacts when the explosive detector would be dropped down to the hard surface.

In yet another embodiment, the front part of the IR sampler 1, created according to the points [0062-0067] is equipped with the cone-shaped hood 8 (FIG. 2, 5), creating thus the pneumatically closed area for successful sampling of the explosive traces from the scanned surface.

In another embodiment, the cone-shaped hood 8 (FIG. 2, 5) is made from highly chemically inert and temperature resistant material “PEEK”.

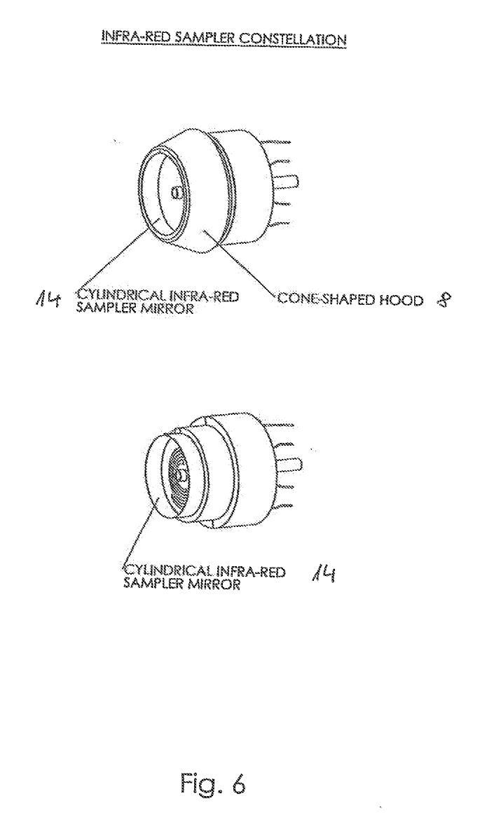

In yet another embodiment, the inner cylindrical surface of the cone-shaped hood 8 (FIG. 6) is equipped with the highly polished stainless-steel cylindrical IR sampler mirror 14 to improve the homogeneity of the areal Infra-Red radiation and for increasing the effectiveness of the radiation.

In yet another embodiment, the completed formation of the Infra-Red sampler 1 together with thermal decomposition unit 2 (FIG. 2) is mounted directly to the terminal flex-part of the main PC board without any connectors to secure the highest possible reliability of the system.

Analytical and Detection System

As shown in the block diagram provided in FIG. 1 and drawings at FIG. 2, once the traces of explosives from the scanned surface are released and converted to the vapor phase using the Infra-Red radiation energy, the obtained gas sample is directly sucked by integrated vacuum pump 15 (housed in the neoprene pump lodgement 16), into the thermal decomposition unit 2 (FIG. 2), having the gas input located directly in the center of the flat Infra-Red radiation source 5 (FIG. 2, 5). In this configuration, all the parts operate at the accurate temperature to avoid any condensation of the sampled components and features no loses of the sample as the detection/analytical part is in fact housed just behind the flat Infra-Red radiation source 5 (FIG. 2) and kept at the accurate operational temperature all the operational time.

In exemplary embodiment, the thermal decomposition unit 2 is composed by the silica-glass tube with processed inner surface and the non-linear heating element located on the outer surface in the middle of the length of the tube.

In yet another embodiment, the heating element is composed by a heating wire, wound in non-linear way along the axes of the tube, compensating thus opposite non-linear temperature drops given by location of the tube inside the temperature insulating cylinder 11, to achieve gradient-less distribution of the operating temperature along the decomposition tube.

In yet another embodiment, the heating wire is driven using PWM mode for precise regulation of the thermal decomposition unit 2 (FIG. 2) temperature.

In yet another embodiment, the heating wire is used as a temperature sensing element during the power pauses of the PWM cycle. This arrangement secures the tightest sensing element coupling to the mass of the silica-glass tube, securing thus the most precise temperature measurement and regulation of the inner tube surface temperature for the most accurate operation of the thermal decomposition unit 2 provided by the integrated control electronic board (unit) 18. The whole formation is housed into the robust AL/Mg body 12.

In yet another embodiment, the heating wire is fixed in it's non-linear displacement and operating position along the tube by a special high-temperature resistant cement.

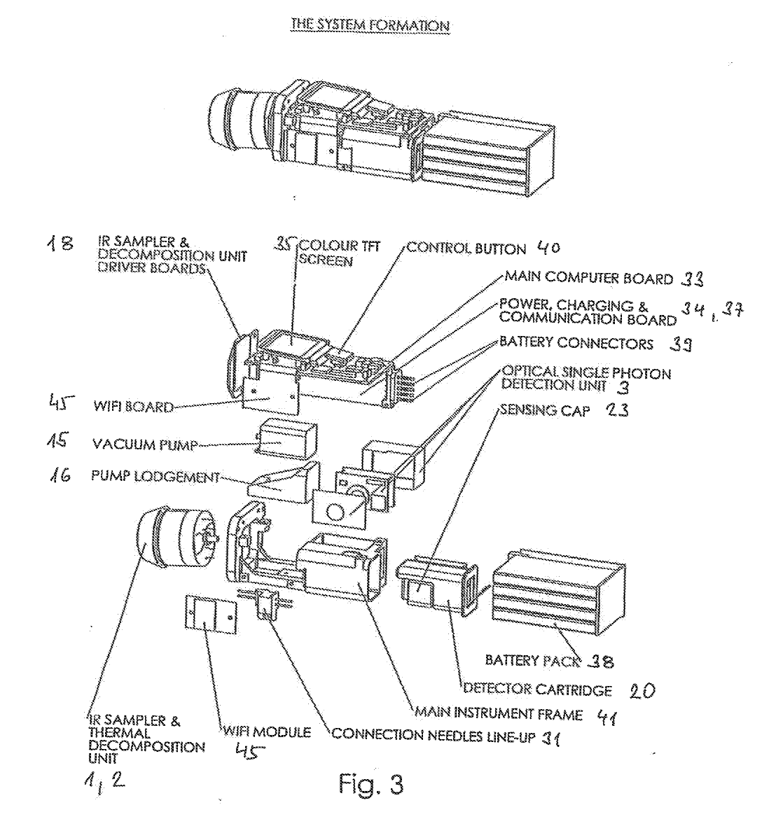

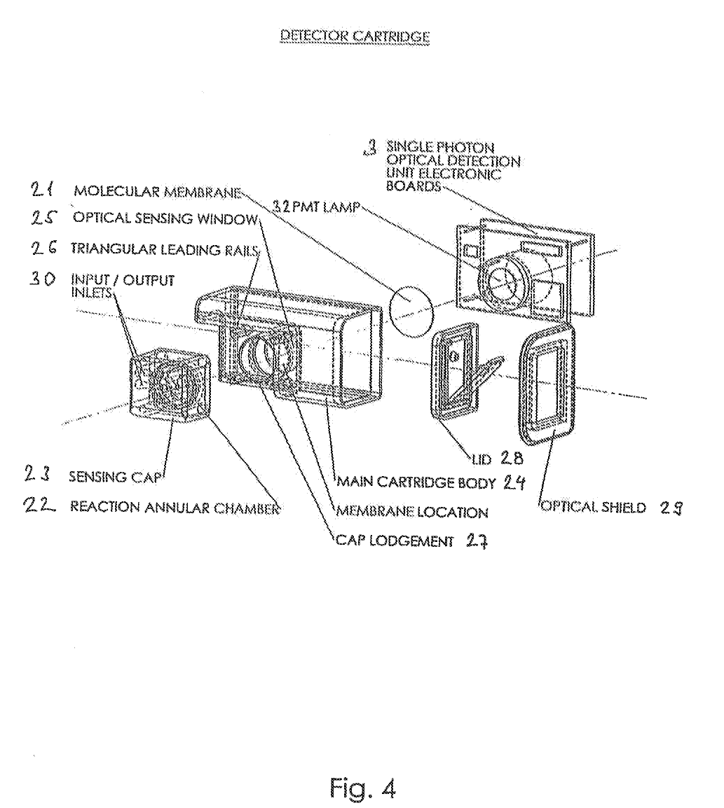

The decomposed sample is further transferred into the analytical unit 19, consisting of detector cartridge 20 (FIG. 1, 4) with in-detection-liquid sunk molecular membrane 21 (FIG. 4), reaction annular chamber 22 (FIG. 4) created inside the sensing cap 23 (FIG. 3, 4) and the optical single photon detection unit 3 (FIG. 4).

In exemplary embodiment the detector cartridge 20 (FIG. 4) consists of the main cartridge body 24, made from transparent and high pH resistant plastics, having the shape according to the invention, shown in details at the FIG. 4.

As shown at FIG. 4, the detector cartridge 20 is built in vertical orientation, having the place of the detection membrane 21 located at the left side (FIG. 4), with the position securing the maximal range of operating explosive detector angles, in which the membrane 21 is fully sunk under the level of detection liquid. The fully sunk membrane 21 (FIG. 4) represents the fundamental condition for the proper operation of the whole detection system and it's detection stability.

As also shown at FIG. 4, the detector cartridge 20 is equipped with the optical sensing window 25, located at the opposite site to the sunk site of the membrane 21 to minimize the length of the optical pass for transferring the generated photons to the optical sensing element of the single photon detection unit 3 (FIG. 4).

As visible at FIG. 4, the detection cartridge is equipped with four triangular leading rails 26 for inserting the sensing cap 23, cap lodgement 27 for sealed positioning of the sensing cap 23, and the covering back lid 28 together with optical shield 29.

In exemplary embodiment, the sensing cap 23 is embattled using the leading rails 26 onto the left side of the detector cartridge 20, providing thus the connection between the thermal decomposition unit 2 (FIG. 2) and the detection membrane 21 (FIG. 4) by means of the input/output inlets 30, and, also the reaction annular chamber 22 (FIG. 4), featuring the proper volume for the adequate gas lavation around the dry membrane surface, to achieve the requested system time constant and operational dynamics.

In another embodiment, the sensing cap 23 (FIG. 4) is manufactured from the flexible plastics, securing the adequate gas and liquid sealing of the cap 23 along it's lodgment 27 (FIG. 4), created at the left side of the detector cartridge 20 (FIG. 4) and being chemically resistance against the measured media and/or the detection liquid.

In yet another embodiment, the sensing cap 23 contains two input/output inlets 30 for connecting the gas input/output connection needles line-up 31. (FIG. 3)

The single photon detection unit 3 (FIG. 4) consists of especially developed sub-miniature photomultiplier (PMT) lamp 32, sensing analogue electronics, high voltage source and control processor, that controls the operation of the whole single photon detection unit 3 and provides the first level of the intelligent digital signal processing, converting the photomultiplier analogue signal to the digital stream, with digital noise reduction and automatic baseline handling.

The digital output of the single photon detection unit 3 (FIG. 4) is further processed by the system computer located at the main processor and interface board 33, which right side part contains the power, charging and communication board 34 (FIG. 3), with the output, displayed at the color screen 35 (TFT display) (FIG. 3) either in numerical and graphic form and, also, stored at the main system flash disk 36. The main processor and interface board 33 is connected by flex part the power unit 37 and main battery pack 38 (connected by high current battery connectors 39) supplying the whole electronic system. The operating of the system is manually controlled by operator by pressing the control button 40 (FIG. 3) located at the upper part of the whole explosive detector. The whole constellation of above described electronic boards are located inside the unit by fixing along the main instrument plastic frame 41. (FIG. 3, 8). The completed explosive detector is equipped with plastic holster 46 protecting the explosive detector and securing comfortable wearing the unit by operator.

Calibration of the Analytical System

The detection and analytical system according to the invention is calibrated using a especially developed calibration device in lieu of certified standards by exposing the detection and analytical system to the known concentration of the known explosives, such as through well characterized solid particles. The especially developed calibration device works using the principle of computer controlled continues thermal vapor phase generator, creating defined flow of defined concentration of the explosive sample in defined number of consequent steps of concentrations, covering thus the whole dynamic range of the detection and analytical system. Multiple thermal vapor phase generators are consequently used to cover the whole wide spectrum calibration for all the existing explosive materials, while each of the generator is used principally just and only for one explosive compound to avoid cross-contamination of the individual thermal vapor phase generator. The calibration system according to the invention is automatically controlled together with actual device just being calibrated by the external computer and the whole calibration procedure is completely robotized, including automatic saving of the calibration results onto the calibrated analytical system flash disk.

Calibration of the Infra-Red Sampler

The properly operating Infra-Red sampler 14 (FIG. 2) provides the important first level of rough pre-separation, using the adequate Infra-Red energy with the homogeneous flow of radiation to release the solid explosive traces from the scanned subject. To achieve the successful operation of the Infra-Red sampler 14 with well operating collection of explosive traces, the accurate calibration of the sampler/Infra-Red source 5 is necessary.

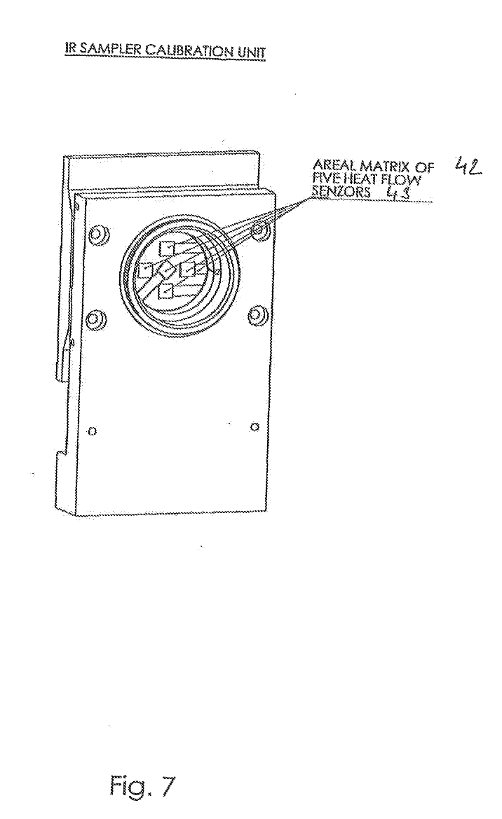

The special calibration device (FIG. 7) for the Infra-Red source 5 has been developed. According to the invention, it is composed by the Infra-Red flow detection chamber, using the matrix 42 of five units of heat flow sensors 43 (refer to FIG. 7), displaced in the center and surrounding the center of the IR exposed area. The matrix 42 of five heat flow sensors 43 measure the heat flow intensity and area distribution of the flow.

According to the invention, the autonomously operated calibration software of the calibration device automatically sets up the parameters of the Infra-Red sampler 14 to achieve the correct spectrum of the Infra-Red radiation and correct heath flow for proper operation of the sampler 14 and automatically stores the data to the analytical system flash disk 36.

Accordingly, the above description should not be as pertaining only to the precise structures described and illustrated in the accompanying drawings, but rather should be read consistent with and as support to the following claims, which are to have their fullest and fair scope.

### LEGEND

1 IR Sampler
2 Thermal decomposition unit
3 Optical detection unit
4 Control unit
5 Infra-Red radiation source
6 Helix-shaped radiation element
7 Black daze flat cover/shield
8 Hood
9 Aperture
10 Inlet
11 Insulation cylinder
12 Al/Mg body
13 Skin
14 IR sampler mirror
15 Pump
16 Pump lodgement
17 Communication unit
18 Control electronic board
19 Analytical unit
20 Detector cartridge
21 Molecular membrane
22 Annular chamber
23 Sensing cap
24 Cartridge body
25 Optical window
26 Leading rails
27 Cup lodgement
28 Lid
29 Optical shield
30 Input/Output inlets
31 Needles line-up
32 PMT lamp
33 Main processor and interface board
34 Power, charging ad communication board
35 Screen
36 Flash disk
37 Power unit
38 Battery pack
39 Battery connectors
40 Control button
41 Instrument frame
42 Areal matrix
43 Sensors
44 USB and RS 232 ports
45 WiFi module
46 Holster

What is claimed is:

1. Compact size explosive detector with ultra fast response and high sensitivity, comprising:
an Infra-Red sampler (1) having a flat Infra-Red radiation source (5), and
a thermal decomposition unit (2), having the gas input located in the center of the flat Infra-Red radiation source (5) and a silica glass tube with heating element, capable of decomposing each component into a plurality of molecular fragment, and
a detector cartridge (20), having the main irregularly shaped rectangular body (24) with special detection liquid inside, a molecular detection membrane (21), a sensing cap (23), and black rear rectangular lid (28), where each molecular fragment is detected by said detector, and
a single photon optical detection unit (3) in communication with a Infra-Red sampler (1) and detector cartridge (20), to provide data stream indicative of explosives material presence in the sample, and
a main processor and interface board (33), integrating all the instrument electronics including the main control and processing computer, power and charging blocks, color display driver, analogue measurement and power circuits for controlling the analytical section, providing the numerical as well as graphical output of the analyses, storing the data to the system flash disk (36) and controlling the operation of the system.

2. The compact size explosive detector with ultra fast response and high sensitivity of claim 1, wherein the Infra-Red sampler (1) further comprising a flat Infra-Red radiation source (5), said source emitting the Infra-Red energy of proper wavelength homogeneously from it's flat surface to the scanned subject, said source being created by modified helix-shaped Infra-Red radiation element (6) with gradient-compensation loops at the lower part, said element being covered by two-layers black thin-film daze shield (7), dismissing mainly Infra-Red radiation, said shield having the central circular aperture (9) for interposition of the sampling silica glass inlet, the completed formation of the said parts being integrated into the one body with the temperature insulating cylinder (11) housed in molded aluminium/magnesium body (12), said source being equipped with cone-shaped hood (8) made of highly temperature resistant plastic, said hood being equipped with highly polished stainless-steel cylindrical mirror (14) for homogeneous and effective Infra-Red radiation flow.

3. The compact size explosive detector with ultra fast response and high sensitivity of claim 1, further comprising a thermal decomposition unit (2), having the gas input located in the center of the flat Infra-Red radiation source (5), being composed by silica glass decomposition tube, said tube being equipped with non-linear heating element located at tube outer surface in the middle of the tube length, said heating element being composed by heating wire wound with non-linear spacing to compensate for temperature loses at both ends of the tube and for securing gradient-less distribution of the operating temperature along the length of the decomposition tube, said heating element being driven by PWM driver to achieve effective power management and accurate temperature measurement, said heating wire is used as the temperature sensing element with the temperature precisely measured by measuring the resistance of the heating wire during the pauses of the PWM power cycle, while the temperature of the heating wire is almost identical with the temperature of the inner surface of the decomposition tube due to tight temperature coupling of the heating wire to the decomposition tube, said heating element with said defined non-linear coil spacing is fixed to the outer silica glass tube surface by special high temperature resistant cement, the said formation of the temperature decomposition unit (2) is housed inside the said temperature insulating cylinder (11) and having the gas inlet located in the middle to the flat Infra-red radiation source (5).

4. The compact size explosive detector with ultra fast response and high sensitivity of claim 1, wherein further comprising detector cartridge (20), having the main irregularly shaped rectangular body (24) with special detection liquid inside, a molecular detection membrane (20), a sensing cap (23), and black rear rectangular lid (28), the main body (24) being made of transparent and highly pH resistant plastic, having the cylindrical lodgment (27) and triangular leading rails (26) for the sensing cap (23) on the left side, together with the rim, created as an extension of the said circular lodgment (27) for molding of the detection molecular membrane (21), said main body (24) having also the polished optical sensing window (25) on the opposite right site to the location of the detection molecular membrane (21), all integrated into one body (24) made by plastic molding, said main body also having the black rear rectangular plastic lid (28) with pulling handle located at the rear side of the said main body, said detector cartridge (20) having also the sensing cap (23) made of flexible plastics, embattled from the left side on the cylindrical detection cap lodgment (27) of the detector cartridge (20), with proper position given by triangular leading rails (26), said cap (23) containing two input/output inlets (30) for connecting gas input/output connection needle line-up (31), annular reaction chamber (22) for the analyzed gas circulating around the outer left dry surface of the molecular detection membrane (21) and cylindrical sealing part fitted with negative diameter tolerance to the cylindrical cap lodgment (27) of the detector cartridge (20).

5. The compact size explosive detector with ultra fast response and high sensitivity of claim 1, further comprising a single photon optical detection unit (3) having an especially developed sub-miniature photomultiplier lamp (32), sensing analogue electronics, high voltage source and control processor, said control processor running the program being created to provide intelligent first level signal processing converting the photomultiplier lamp (32) signal to the digital data stream with advanced digital noise reduction procedure and compensation for the photomultiplier lamp's non-linear operation characteristics, temperature dependences and automatic base-line handling.

6. The compact size explosive detector with ultra fast response and high sensitivity of claim 1, further comprising a main processor and interface board (33), integrating all the instrument electronics including the main control and processing computer, power and charging blocks, color screen driver, analogue measurement and power circuits for controlling the analytical section, all being realized in the form of multiple individual boards (33, 34, 37), connected by flex strips connection boards creating thus one multiple board body (33, 34), together being installed in 3-D rectangular positions around the main instrument frame (41) without the need of any cross-connecting connectors, the said multiple board (33, 34) mounted in rectangular 3-D shape around the main instrument frame (41), giving the instrument construction the exceptional mechanical rigidity and robustness, the said multiple board being constructed for one-clip optional mounting of the WiFi wireless communication module (45) the said multiple board (33, 34, 37) having the integrated rear battery connectors (39) for connecting the exchangeable battery pack (38).

7. The compact size explosive detector with ultra fast response and high sensitivity of claim 1, wherein the explosive detector further comprises a stored calibration data for either quantitative and qualitative analyses of the sample, created by automatic calibration device.

8. The compact size explosive detector with ultra fast response and high sensitivity of claim 7, wherein the automatic calibration device works using the principle of computer controlled continues thermal vapor phase generators, creating defined flow of defined concentration of the explosive sample in defined number of consequent steps of concentrations, covering thus the whole dynamic range of the detection and analytical system.

9. The compact size explosive detector with ultra fast response and high sensitivity of claim 8, wherein the multiple thermal vapor phase generators are consequently used to cover the whole wide spectrum calibration for all the existing explosive materials, while each of the generator is used principally just and only for one explosive compound to avoid cross-contamination of the individual thermal vapor phase generator.

10. The compact size explosive detector with ultra fast response and high sensitivity of claim 7, wherein the automatic calibration devices are automatically controlled together with actual compact size explosive detector just being calibrated, by the external computer and the whole calibration procedure is completely robotized, including automatic saving of the calibration results onto the calibrated explosive detector's flash disk (36).

11. The compact size explosive detector with ultra fast response and high sensitivity of claim 1, wherein the Infra-Red sampler (1) provides the important first level of pre-separation, using the adequate Infra-Red energy with the homogeneous flow of radiation to release the solid explosive traces from the scanned subject.

12. The compact size explosive detector with ultra fast response and high sensitivity of claim 1, wherein the calibration device for the Infra-Red source (5) is composed by the Infra-Red flow detection chamber, using five units of heat flow sensors (43), displaced in the center and surrounding the center of the IR exposed area to measure the heat flow and area distribution of the flow.

13. The compact size explosive detector with ultra fast response and high sensitivity of claim 12, wherein the calibration device for the Infra-Red source (5) includes the autonomously operated calibration software for automatically setting up the parameters of the Infra-Red sampler (1) to achieve the correct spectrum of the Infra-Red radiation and to correct heath flow for proper operation of the sampler (1) and to automatically store the data to the analytical system flash disk (36).

14. The compact size explosive detector with ultra fast response and high sensitivity of claim 1, wherein the explosive detector further comprises a signal processing algorithm for processing a multiparameter's data stream.

15. The compact size explosive detector with ultra fast response and high sensitivity of claim 1, wherein the explosive detector has a case, designed to fit organically to the operator's hand for secure, errorless and comfortable operation.

16. The compact size explosive detector with ultra fast response and high sensitivity of claim 1, wherein the explosive detector's operating system is designed to be operated as simple as possible using just one control button (40) and transparent and easy-to-understand menu.

17. The compact size explosive detector with ultra fast response and high sensitivity of claim 1, wherein the explosive detector's operational parameters may only be changed by comfortable PC software protected by password.

18. The compact size explosive detector with ultra fast response and high sensitivity of claim 1, wherein all the operations of the explosives detector may be remotely controlled and measured data downloaded between PC or mobile ANDROID platform via wire (USB or RS 232 ports (44)) or wire-less via installed WiFi module (45) or via Internet connection.

19. The compact size explosive detector with ultra fast response and high sensitivity of claim 1, wherein the explosives detector can be fully diagnosed and first-level repaired remotely via Internet from manufacturer's site.

20. The compact size explosive detector with ultra fast response and high sensitivity of claim 1, wherein the explosive detector is equipped with operator's belt plastic holster (47) for comfortable whole-day wearing and dexterous operation.

21. A method for detecting explosives comprising:
Infra-Red release of the solid explosive samples from the surface of the scanned subject using the dedicated wavelength of the Infra-Red radiation,
thermal decomposition of the obtained gas sample,
converting the decomposed molecule's concentration to the optical signal,
analyzing the shape of time peak of the optical signal to distinguish interfering components from the signal of interest and to remove wide spectrum noise and signal fluctuations of various origin and
analyzing the data obtained from above signal processing, displaying the data in numerical and/or graphic form on the instrument color graphic screen and storing the data to the system flash disk.
